(19) United States
(12) Patent Application Publication
(10) Pub. No.: US 2012/0040328 A1
(43) Pub. Date: Feb. 16, 2012

(54) FERMENTER FEED SYSTEM FOR FERMENTABLE BIOMASS OF A BIOGAS SYSTEM AND METHOD FOR OPERATING THE FEED SYSTEM

(76) Inventor: Michael Niederbacher, Bruneck (IT)

(21) Appl. No.: 13/138,292

(22) PCT Filed: Jan. 27, 2010
(86) PCT No.: PCT/EP2010/000497

§ 371 Date: Sep. 21, 2011

(30) Foreign Application Priority Data
Jan. 28, 2009 (EP) 09001148.7

(51) Int. Cl. C12Q 3/00; C12M 1/38

(52) U.S. Cl. 435/3; 435/286.6

(57) ABSTRACT

A fermenter feed system for fermentable biomass of a biogas system comprising at least one fermenter, and a delivery and treatment device located between a biomass depot and the fermenter. A mechanical cell breakdown apparatus (6) processes the biomass for delivery (5, 10, 11, 14, 34) to the fermenter (18,19) which includes an outlet opening (23) with a diameter. The diameter may be varied by an adjustable actuating member (32) according to at least one sensor (26) for detecting an operational parameter value of the cell breakdown apparatus (6) or the biomass. A diameter regulator (25) receives an output signal from the sensor as an actual parameter value and compares it to a predetermined target value (28). The actuating member (32) is activated when the actual value falls below the target value to make a predetermined change to the outlet opening diameter.

### BACKGROUND OF THE INVENTION

The invention relates to a fermenter feed system for fermentable biomass of a biogas system, as well as a method for operating the feed system.

In commonly known biogas systems, a fermented wet anaerobic breakdown process is used to extract biogas biomass. Automated fermenter feed systems are customarily used to feed the fermenter with fermentable biomass, whereby pumps are provided for liquid substrate components. Delivery and treatment devices are provided for solid substrate components, and delivery and treatment devices are provided for solid substrate components, between a solid biomass depot and a fermenter feed port. As a rule, such a delivery and treatment device comprises a dosing feeder as well as conveying means, in particular conveyor belts and/or screw conveyors, leading to the fermenter. A known dosing feeder consists of a container, for example, that can be filled with biomass, which sits on weighing cells, from which a predetermined biomass amount is fed to the fermenter by means of a floor conveyor belt connected to extruding screws on the face side for further transport to the fermenter.

The biogas produced is either injected directly into the gas pipelines after cleaning, or used for power generation in a thermal power station. The economic effectiveness of a biogas system depends essentially on the type of the biomass that can be injected, its availability, as well as the effectiveness of the fermentation process with the shortest possible substrate dwell time in the fermenter with a high degree of breakdown for a high biogas yield.

Furthermore, a method for the production of plant substrates for the growing of cultivated plants in the garden and landscaping is known (DE 44 44 745 C1) with which the wood waste premixed with sewage sludge, liquid manure and biocompost are broken down, fiberized and vigorously mixed together in a screw extruder, wherein temperatures rise to over 100° C. As a result, pathogens, bacteria, etc. are killed off, so that the largely sterile plant substrate on the extrusion head is discharged for further use. Furthermore, a similar method for the disposal of biological waste from cities and towns is known (DE 195 14 975 A1).

From EP 1 978 086 A1 a process for the hybrid breakdown of water- and lignocellulose-containing biomass by means of defibration and hydrolysis is known, in which in a first stage lignocellulose-containing biomass is treated by a defibration device, and then, in a subsequent stage, the treated, lignocellulose-containing biomass is heated in a thermal hydrolysis process in a high-pressure microwave and hydrolyzed before finally, in a last stage, the hydrolyzed lignocellulose-containing biomass is subjected to an aerobic fermentation process. Within the thermal hydrolysis of the biomass in a high-pressure microwave, the water-containing biomass is briefly heated to far over 100° C., so that through high temperatures and the evaporated water from the biomass, thermal hydrolysis takes place. The temperature rise thereby takes place from 160 to 220° C., for example, with a great increase in pressure from 5 to 25 bar. The defibration of the biomass is preferably carried out by means of an extruder, which may be designed as a single screw extruder or a twin-screw extruder or a twin-worm extruder. With this type of process, a shortening of the customary dwell time and fermentation time in the fermenter of a biogas reactor should be achieved in addition to the increase in biogas yield. This type of design is relatively energy-intensive and also expensive to manufacture, in particular due to the use of a high-pressure microwave.

### SUMMARY OF THE INVENTION

An object of the invention is thus to propose a fermenter feed system and a method for operating this type of fermenter feed system, wherein the fermenter feed system is simply constructed and can be operated with a low energy cost. The fermenter feed system can be used with a wide range of types of biomass for biomass production and thus can be simply processed in such a way that the subsequent fermentation process can run efficiently and reliably, even with high dry substance content with short substance dwell times and a high degree of breakdown and thus a higher biogas yield.

This objective is achieved according to one embodiment of the invention by providing a fermenter feed system for fermentable biomass of a biogas system which has a delivery and treatment device between a biomass depot, preferably designed as a traveling silo, and which has at least one fermenter, in particular at least one fermenter feed port, whereby the delivery and treatment device has at least one conveying means for the biomass to be fed to the fermenter. Furthermore, a preferably mechanical cell breakdown apparatus for biomass is integrated into the feed and treatment device, via which at least a part of the biomass to be delivered to the fermenter is conducted and is fed to the fermenter after the cell breakdown.

Due to such cell breakdown, biomass solids can advantageously be used during the biogas production in the fermentation process, particularly straw and grass, which otherwise are not fermentable or are only ineffectively fermentable. Furthermore, a reduction in size of the biomass solids occurs with the cell breakdown, so that these are more accessible for the bacteria in the fermenter and thus in the fermentation process, and form a structure which is more easily attacked, so that a high dry substance content in the substrate with lower dwell times and higher biogas yield is possible. In addition, savings can thus be achieved in the dimensioning and operation of the fermenter mixing process, whereby a tendency to form floating layers or sinking layers is reduced. Due to this pretreatment, the biogas yield is considerably increased, which in turn results in low use of biomass and which is reflected in smaller sizes for the fermenter, so that the biogas system is cheaper to purchase and to operate than the customary biogas systems.

According to the invention, the diameter of an outlet opening of the cell breakdown apparatus, preferably the diameter of an outlet opening or nozzle of a cell breakdown extruder, may be varied by way of an adjustable actuating member, in particular an actuating member that can be controlled by an electric, hydraulic or pneumatic drive. Furthermore, at least one temperature sensor and/or pressure sensor is associated with the cell breakdown apparatus by means of which the temperature and/or the pressure in a predetermined region of the cell breakdown apparatus and/or biomass is/are detected. Furthermore, a diameter regulator is provided, to which the output signal of the temperature sensor is fed as an actual temperature value and/or of the pressure sensor as an actual pressure value. The actual value is compared to a predetermined target value, and the regulator is activated when the actual value falls below the target value for a defined, predetermined reduction of the outlet opening diameter.

With the fermenter feed system according to the invention, a desired cell breakdown process can be ensured in a simple and operationally reliable way, and the required temperature and/or pressure required in the cell breakdown apparatus can be ensured. Specifically, the measuring values of the temperature sensor and/or pressure sensor can be arranged, for example, in the proximity of the outlet nozzle of a cell breakdown apparatus, and supplied to the diameter regulator as actual values, which can be set at a target value and/or target pressure for a reliable cell breakdown, particularly a target temperature value default of at least 40° C., most preferably at least 60° C. If there is a trend towards a fall in the actual temperature value and/or of the actual pressure value below the particular predetermined target value, then the actuating member for the reduction of the outlet opening diameter is triggered by the regulator. As a result, for comparable power consumption of the cell breakdown apparatus, more energy is introduced in the current delivered and treated biomass, so that an optimal biomass adapted to this particular case can be made available for a fermentation in a fermenter of a biogas system.

The temperature sensor and/or pressure sensor in the cell breakdown apparatus is/are preferably arranged directly upstream from or in the outlet opening, in particular directly upstream from or in an outlet nozzle of a cell breakdown extruder. For example, it can be provided that the temperature sensor and/or the pressure sensor is arranged in a wall region or in an inner lining region of the cell breakdown apparatus in the proximity of the outlet opening. These possible attachments of the sensors, previously stated as examples, demonstrate that there are various possibilities in the region of the cell breakdown apparatus to detect an actual temperature and/or an actual pressure in order to directly or indirectly deduce whether, in the region of the biomass treated in the cell breakdown apparatus, a desired cell temperature and/or pressure for an optimal cell breakdown exists. An indirect detection the temperature means that it is possible, for example, to measure the temperature in a wall region in the proximity of an outlet opening of the cell breakdown apparatus, on the basis of which the temperature in the treated biomass may be deduced.

The cell breakdown apparatus can be advantageously integrated into the conveying path for the biomass between a dosing feeder and the fermenter. As a result, the cell breakdown apparatus is arranged in the outer region and exposed to the weather conditions. Thus, it is proposed to design a thermally insulated cell breakdown apparatus, either by means of direct thermal insulation in the manner of an insulated casing or indirect thermal insulation in the form of an insulated housing. Such thermal insulation is advantageous and essential in particular, for example, when a cell breakdown extruder with worm shafts, preferably a double worm shaft extruder having a dosing feeder and an outlet nozzle, is used. However, optionally the thermal insulation is sufficient even only if provided in the region of the worm shafts. Cell breakdown takes place with this type of extruder by the introduction of mechanical energy into the biomass conveyed in the extruder by means of friction of the biomass, rotating with the worm screws, against the stationary housing cylinder wall. High pressures and high temperatures can thus be produced in the biomass. Discharge of the biomass at the outlet nozzle then causes a pressure relaxation and/or temperature cooling, which leads to the cell breakdown.

An advantageous formation of the conveying path with the incorporated cell breakdown apparatus, in particular with a cell breakdown extruder, is provided. In the process, a first conveyor, preferably a first belt conveyor, goes from the dosing feeder into a region above the cell breakdown apparatus. A second conveyor, preferably a second conveyor belt, goes out from a region under the outlet opening or outlet nozzle of the cell breakdown apparatus to the fermenter. With the first conveyor belt, biomass can be fed directly to the cell breakdown apparatus, for example, via a feed hopper with a motor-drivable stuffing screw. In the event of a malfunction of the cell breakdown apparatus, due to hard impurities, in particular metal pieces, in order to ensure continued feeding of the fermenter, an activatable damper is preferably provided, by means of which either the cell breakdown apparatus is fed according to the adjusted position, or a bypass directly to the second conveyor belt for direct transport to the fermenter.

Alternatively, a third conveyor belt connected to the first conveyor belt is provided, whose running direction is reversible, so that the cell breakdown apparatus can be fed in the one direction, while the biomass can be transported directly in the other direction, for example, via a pipe socket, in a direct and bypass path to the second conveyor. In this embodiment with a third conveyor, in particular a conveyor belt, the above-explained damper connected with the bypass at that location is unnecessary.

To protect the cell breakdown apparatus, in particular a cell breakdown extruder, and for improved reliability, at least one malfunction detection device for impurities in the biomass, for example, for metal pieces, stones, etc. is provided in the dosing feeder. With substance impurity detection, the above-mentioned damper to the bypass and/or transport direction can be reversed for the third conveyor, whereby this process is preferably automated.

In order to equalize the power consumption, in particular of an electric drive for the cell breakdown apparatus, to a suitable level, a regulation of the extruder feed quantity via a feed regulator is proposed whereby this regulation acts on a rotationally driven stuffing screw. For this purpose, the power consumption is measured as the actual value and, for example, for power consumption which is predetermined or which tends to decrease, the screw conveyor speed is increased via the feed regulator for a greater biomass feed. This regulator is appropriately designed via suitable control parameters for a quicker control response in comparison to the nozzle diameter control of the outlet opening.

The object regarding the method is achieved by the features of claim 11. The resulting advantages have already been increased in connection with the advantages for the fermenter feed system.

### DESCRIPTION OF THE DRAWINGS

The present invention shall now be explained in more detail by reference to several preferred embodiments thereof, schematically represented in the drawings.

FIG. 1 is a perspective view of a fermenter feed system according to the invention;

FIG. 2 is a top view of the fermenter feed system according to FIG. 1; and

FIG. 3 is a cell breakdown extruder illustrated as a twin-screw extruder.

### DESCRIPTION OF A PREFERRED EMBODIMENT

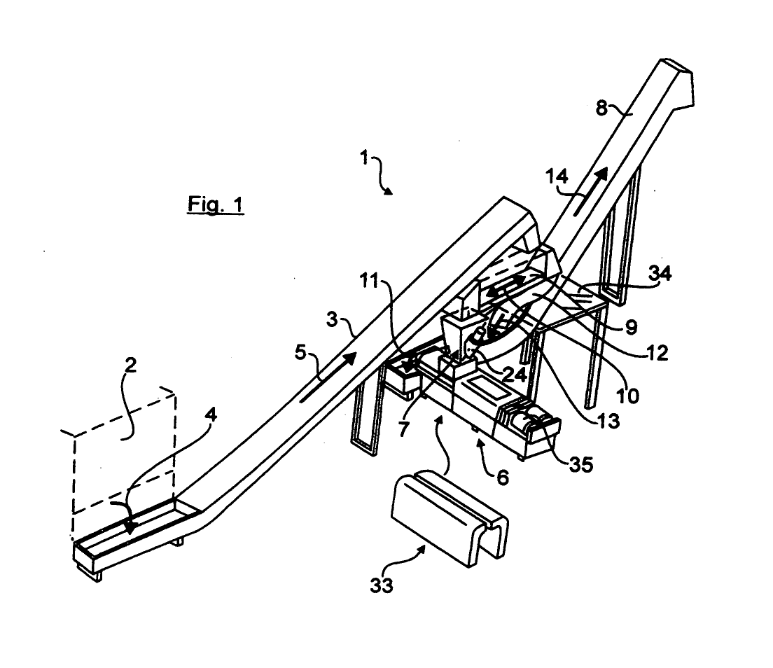

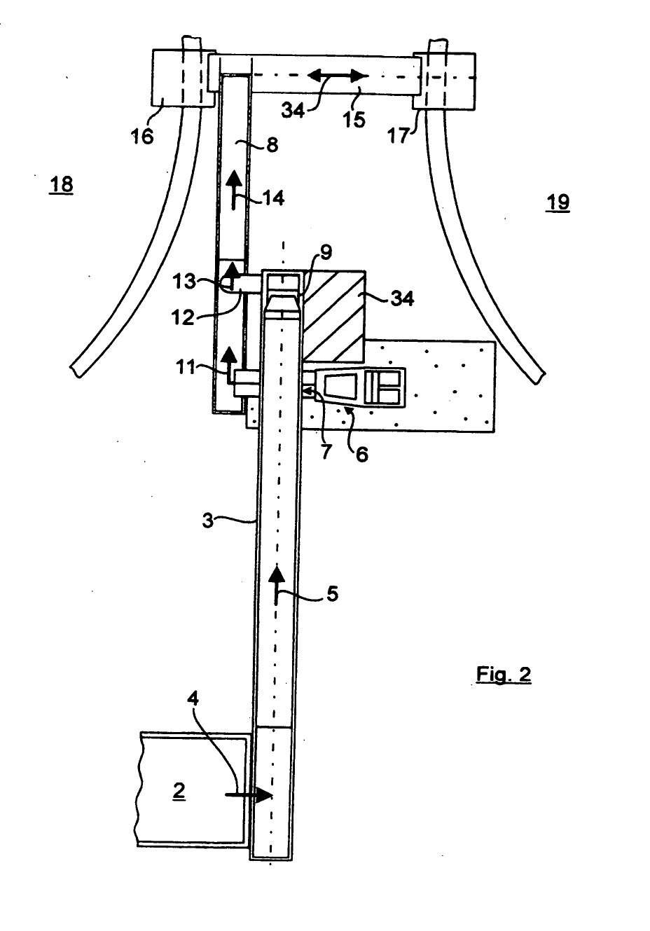

FIGS. 1 and 2 show a fermenter feed system 1 in a perspective top view, whereby a dosing feeder 2 (indicated only schematically) is arranged downstream from a biomass depot (not shown) as a traveling silo.

Weight-controlled biomass is fed from dosing feeder 2 to a first conveyor belt 3 (Arrow 4), by means of which the biomass is conveyed upwards (Arrow 5), specifically, into a cell breakdown extruder 6 which forms a cell breakdown apparatus. Under the upper end of the first conveyor belt 3 and above a cell breakdown feeder 7, particularly above a breakdown extruder 6. A third conveyor belt 9 is attached which cooperates, directly or indirectly via the breakdown feeder 7, with a second conveyor belt 8. Preferably the breakdown feeder is a type known as a hopper feeder.

For this purpose, the conveying direction of the third conveyor belt 9 can be reversed (Double arrow 10), so that (in normal operation) biomass, which is transported from the first conveyor belt to the third conveyor belt, is supplied via the breakdown feeder 7 to the cell breakdown extruder 6, and following the treatment there, is fed to the second conveyor belt 8 (Arrow 11). With a reversal of the running direction of the third conveyor belt 9, for example after an automated recognition of impurities in the conveyor path to the cell breakdown extruder 6, the biomass reaches the region of the beginning of the belt of the second conveyor belt 8 (Arrow 13) in a direct path via a conveying tube 12 as bypass through free fall.

With the second conveyor belt 8, treated biomass from the cell breakdown extruder 6 (Arrow 11) or biomass is transported upwards (Arrow 14) via the direct path, specifically, via a transverse fourth conveyor belt 15, which runs between two fermenter feed connectors 16, 17, whereby only a partial section of a fermenter wall of the associated adjacent fermenter (18, 19) is shown (see FIG. 2). The running direction of the fourth conveyor belt 15 can be reversed (Double arrow 34), so that, depending on the running direction, fermenter 18 or fermenter 19 is fed with biomass.

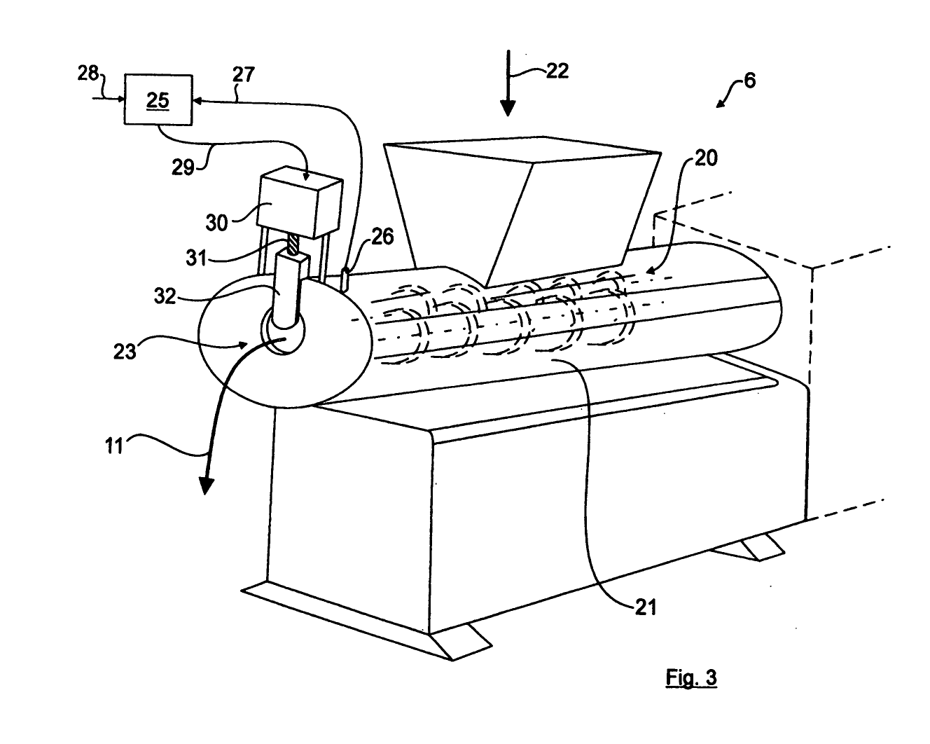

In FIG. 3, a schematic drawing of the front part of the cell breakdown extruder 6 depicts a twin-screw extruder in which the interacting twin-screw extruders 20 are driven and arranged in a narrow screw housing 21 in such a way that fed biomass (Arrow 22) is conveyed outward, with a high pressure build-up and a high temperature increase, against and through an outlet nozzle 23 (Arrow 11), where the defibrated and frayed biomass is again relaxed and cooled with cell breakdown.

The structure of the breakdown feeder (not yet installed in FIG. 3) can be seen in FIG. 1 and comprises here, for example, a hopper feeder 7 with an associated stuffing screw 24. The stuffing screw 24 is thereby rotationally driven in a controlled manner, so that the biomass feed (Arrow 22) is metered quantitatively to the cell breakdown extruder 6 in such a way that the electric drive motors 35 for the twin screws 20 are operated essentially with equal power, advantageously in an upper effective power region.

The diameter of the outlet nozzle 23 is designed here with a diameter regulator 25 so that it is temperature-controlled, since for cell breakdown a cell temperature of the biomass should reach at least 65° C. in the screw housing 21 upstream from the outlet nozzle 23. A temperature sensor 26 is thus provided in this region as the actual temperature value sensor whose actual value signal is fed to the diameter regulator 25 (Arrow 27). As the target value (Arrow 28) a temperature of 70° C., for example, is set at the diameter regulator 25, which is compared with the actual temperature value. If there is a control deviation, the diameter regulator 25 emits a control signal (Arrow 29) to a servomotor 30, which by means of a spindle drive 31 displaces a slider 32 in the outlet opening of the outlet nozzle 34 in such a way that when the actual temperature value falls below the set target temperature, the slider 32 is inserted further into the outlet opening of the outlet nozzle 23 to reduce the diameter.

Since the cell breakdown extruder 6, as a component of the relatively large fermenter feed system 1, is to be set up outdoors, and a high temperature upstream from the outlet nozzle 23 in the region of the twin-screw 20 is required for cell breakdown, this region in particular is designed so that it is thermally insulated. For this purpose, the insulation half-shells 33, made of insulating material, depicted in FIG. 1 and having the required cutouts for the breakdown feeder 7, are placed on the screw housing 21, or optionally on the entire extruder for thermal insulation thereof. As a result, if there is a change in temperature, the extruder operation is equalized and the regulation is stabilized, whereby the power requirement of the cell breakdown extruder 6 is also advantageously reduced in comparison to a lack of thermal insulation. As additional weather protection, the extruder region can be further covered (not shown). Furthermore, for installation and maintenance purposes a platform (34) accessible by ladders (not shown) can be attached at the height of the third conveyor belt 9.

While a preferred embodiment of the invention has been described using specific terms, such description is for illustrative purposes only, and it is to be understood that changes and variations may be made without departing from the spirit or scope of the following claims.

1. A fermenter feed system for a fermentable biomass of a biogas system comprising:
at least one fermenter, a delivery and treatment device located between a biomass depot and said at least one fermenter, said delivery and treatment device comprising at least one conveyor for moving biomass to said at least one fermenter feed connector (16, 17) of said fermenter (18, 19), a mechanical cell breakdown apparatus (6) disposed between said depot and fermenter for processing said biomass prior to delivery to the fermenter (18,19), and said processed biomass (5, 10,11,14, 34) being delivered to the fermenter (18,19) after the cell breakdown so that the processed biomass is further integrated into the delivery and treatment device, wherein:
said cell breakdown apparatus (6) includes an outlet opening (23) with a diameter which may be varied by an adjustable actuating member (32);
one of a temperature sensor and a pressure sensor (26) for detecting one of a temperature and pressure in one of a region of the cell breakdown apparatus (6) and the biomass; and
a diameter regulator (25) for receiving of an output signal from one of the temperature sensor (26) as an actual temperature value (27) and a pressure sensor as an actual pressure value, said regulator comparing said actual value to a predetermined target value (28), and said actuating member (32) being activated when the actual value falls below a target value to make a predetermined change of the outlet opening diameter.

2. The system of claim 1 wherein said actuator member includes a controllable motor selected from one of an electric motor, a hydraulic drive, and a pneumatic drive.

3. The system of claim 1 wherein said one of said temperature sensor (26) and said pressure sensor in the cell breakdown apparatus (6) is arranged directly upstream in said outlet opening (23) of said cell breakdown extruder.

4. The system of claim 1 wherein the cell breakdown apparatus includes direct thermal insulation for outdoor use in the manner by providing one of a thermal insulation casing (33) and a thermal insulation housing.

5. The system of claim 1 wherein the cell breakdown apparatus includes a cell breakdown extruder (6) with screw shafts (20).

6. The system of claim 5 wherein screw shafts include a twin-screw extruder, and having a breakdown feeder (7) and an outlet nozzle as the opening outlet (23).

7. The system of claim 5 including the thermal insulation externally attached to said region of said screw shafts.

8. The system of claim 1 wherein the delivery and treatment device has an upstream crushing and mixing apparatus having a biomass dosing feeder (2).

9. The system of claim 1 wherein said delivery and treatment device includes a biomass dosing feeder, a first conveyor (3) leading from said dosing feeder to a region above the cell breakdown apparatus (6), a second conveyor (8) disposed generally under said outlet opening of said breakdown apparatus, said second conveyor leading directly or indirectly to the fermenter breakdown connector (16, 17), said first conveyor (3) being disposed above a breakdown feeder (7).

10. The system of claim 9 wherein said breakdown feeder includes a hopper feeder having a motor-drivable stuffing screw (24), a control damper, and a bypass to the second conveyor (8) so that said biomass for the feed hopper can bypass the cell breakdown apparatus and be fed directly to the second conveyor (8) for transport into the fermenter depending on the position of the control damper

11. The system according to claim 10 including a third conveyor (9) wherein a conveyor end of the first conveyor (3) lies in a middle region above said third conveyor (9), and said third conveyor (9) can transport said biomass in a transport direction (10) to said hopper feeder (7) via a direct path (2) to the second conveyor (8) whereby optionally the control damper and the bypass attached thereto are dispensed with.

12. The system of claim 10 wherein said breakdown feeder (7) is associated with at least one impurities recognition device for detecting impurities in the biomass, wherein the control damper to the bypass path and the transport direction (10) for the third conveyor (9) are reversible upon the detection of impurities.

13. The system according to claim 1 wherein said breakdown feeder includes a feed hopper for feeding biomass to said breakdown apparatus having a stuffing screw (24) which is rotationally drivable by a motor control using one of an electric drive, hydraulic drive, and a pneumatic drive, a feed regulator having a power meter for electrical power received from worm gear motors (35), as an actual power value sensor, a power target value sensor, and the rotary drive of the stuffing screw (24) as the actuating member, whereby more biomass can be fed to the cell breakdown extruder (6) for specified power consumption which is decreasing compared to the set power target value, by increasing the rotational speed of the stuffing screw.

14. A fermenter feed system for a fermentable biomass of a biogas system comprising:
at least one fermenter,
a biomass depot for supplying biomass to said fermenter
a delivery and treatment device located between said biomass depot and said at least one fermenter,
said delivery and treatment device comprising at least one conveyor for moving biomass to a feed connector (16, 17) of at least one said fermenter (18, 19),
a mechanical cell breakdown apparatus (6) disposed between said biomass depot and fermenter for processing said biomass prior to delivery to the fermenter (18,19), said delivery and treatment device delivering said processed biomass (5, 10,11,14, 34) to the fermenter (18,19) after the cell breakdown so that the processed biomass is further integrated into the delivery and treatment device,
said cell breakdown apparatus (6) having an outlet opening (23) with a diameter which may be varied by an adjustable actuating member (32);
a sensor (26) for detecting a parameter value at one of a region of the cell breakdown apparatus (6) and the biomass; and
a diameter regulator (25) for receiving an output signal of sensor (26) as an actual parameter value (27), said regulator comparing said actual value to a predetermined target value (28), and said actuating member (32) being activated when the actual value falls below the target value to make a predetermined change of the outlet opening diameter.

15. The system of claim 14 wherein said actuator member includes a controllable drive motor.

16. The system of claim 14 wherein the cell breakdown apparatus includes a cell breakdown extruder (6) with at least one screw shaft (20) having a breakdown feeder (7), and an opening outlet (23).

17. The system of claim 14 wherein the delivery and treatment device includes a biomass dosing feeder (2).

18. The system of claim 17 wherein said delivery and treatment device includes a first conveyor (2) leading from said dosing feeder to a region above the cell breakdown apparatus (6), a second conveyor (8) disposed under said outlet opening of said breakdown apparatus generally leading, directly or indirectly, to a fermenter breakdown connector (16, 17), said first conveyor (2) being disposed above a feed hopper (7) of said breakdown apparatus having a motor-drivable stuffing screw (24), a control damper, and a bypass to the second conveyor (8) so that said biomass for the feed hopper can bypass the cell breakdown apparatus and be fed directly to the second conveyor (8) for transport to the fermenter depending on the position of the control damper

19. The system according to claim 18 including a third conveyor (9) wherein a conveyor end of the first conveyor (3) lies in a middle region above said third conveyor (9), and said third conveyor (9) transporting said biomass in a transport direction (10) to said feed hopper (7) via a direct path (2) to the second conveyor (8) whereby optionally the control damper and the bypass attached thereto are dispensed with.

20. The system of claim 17 wherein said feed hoper (7) is associated with at least one impurities recognition device for detecting impurities in the biomass, wherein the control damper to the bypass path and the transport direction (10) for the third conveyor (9) are reversed upon the detection of impurities.

21. The system according to claim 1 including a feed hopper for feeding biomass to said breakdown apparatus having a stuffing screw (24) which is rotationally drivable by a motor control using one of an electric drive, hydraulic drive, and a pneumatic drive, a feed regulator having a power meter for electrical power received from worm gear motors (35), as an actual power value sensor, a power target value sensor, and the rotary drive of the stuffing screw (24) as the actuating member, whereby more biomass can be fed to the cell breakdown extruder (6) for specified power consumption which is decreasing compared to the set power target value, by increasing the rotational speed of the stuffing screw.

22. A method for operating a fermenter feed system according to claim 1 comprising the steps of specifying a temperature target value of at least 60° C. so that when the temperature is less than this specified temperature target value, the diameter of said outlet opening of the breakdown apparatus is reduced by a specified degree, preferably controlled by characteristic tables, in such a way that the actual temperature value is set and regulated to the specified target temperature value.
